(19) United States
(12) Patent Application Publication
(10) Pub. No.: US 2006/0148725 A1
(43) Pub. Date: Jul. 6, 2006

(54) Selective 11beta HSD inhibitors and methods of use thereof

(75) Inventors: David J. Morris, Providence, RI (US); Andrew S. Brem, East Greenwich, RI (US)

Correspondence Address: LAHIVE & COCKFIELD, 28 STATE STREET, BOSTON, MA, 02109, US

(73) Assignees: The Miriam Hospital, Providence, RI (US); Rhode Island Hospital, Providence, RI (US)

(21) Appl. No.: 11/112,723

(22) Filed: Apr. 21, 2005

Related U.S. Application Data
(63) Continuation-in-part of application No. 10/327,566, filed on Dec. 20, 2002.
(60) Provisional application No. 60/564,430, filed on Apr. 21, 2004.
(60) Provisional application No. 60/342,693, filed on Dec. 21, 2001.

(51) Int. Cl. A61K 48/00; A61K 31/704; A61K 31/573; A61K 31/57

(52) U.S. Cl. 514/26; 514/44; 514/177; 514/178; 514/179

(57) ABSTRACT

Methods for treating glucocorticoid associated states using selective 11β-HSD1-dehydrogenase, 11β-HSD1-reductase and 11β-HSD2 dehydrogenase modulating compounds are described.

### RELATED APPLICATIONS

This application claims priority to U.S. Provisional Patent Application Ser. No. 60/564430, filed Apr. 21, 2004. This application is a continuation-in-part of U.S. patent application Ser. No. 10/327,566, filed on Dec. 20, 2002, which claims priority to U.S. Provisional Patent Application Ser. No. 60/342,693, filed Dec. 21, 2001. The entire contents of each of these applications are hereby incorporated herein by reference.

### BACKGROUND

Glucocorticoids are steroid hormones. One example of a common glucocorticoid is cortisol. Modulation of glucocorticoid activity is important in regulating physiological processes in a wide range of tissues and organs. High levels of glucocorticoids may result in excessive salt and water retention by the kidneys, which may lead high blood pressure.

Glucocorticoids play an important role in the regulation of vascular tone and blood pressure. Glucocorticoids can bind to and activate the glucocorticoid receptor (GR) and, possibly, the mineralocorticoid receptor (MR) to potentiate the vasoconstrictive effects of both catecholamines and angiotensin II (Ang II). Tissue glucocorticoid levels are regulated by two isoforms of the enzyme 11β-hydroxysteroid dehydrogenase (11β-HSD). 11β-HSD converts glucocorticoids into 11-keto metabolites that are unable to bind to mineralocorticoid receptors (Edwards C R et al. (1988) Lancet 2:986-9; Funder et al., (1988) Science 242, 583, 585).

### SUMMARY OF THE INVENTION

In an embodiment, the invention pertains, at least in part, to a method for treating a glucocorticoid associated state in a subject. The method includes administering to the subject an effective amount of a 11β-HSD1 reductase inhibitor, e.g., 11-keto-testosterone, 11-keto-androsterone, 11-keto-pregnenolone, 11-keto-dehydroepiandrostenedione, 3α, 5α-reduced-11-ketoprogesterone, 3α, 5α-reduced-11-keto-testosterone, 3α, 5α-reduced-11-keto-androstenedione, 3α-5β-reduced deoxycorticosterone, 3α, 5α-reduced deoxycorticosterone, 3α,5α-reduced progesterone, 3α, 5α-reduced testosterone, or 3α,5α-tetrahydro-11β-dehydro-corticosterone.

In another embodiment, the invention pertains, at least in part, to a method for treating a glucocorticoid associated state in a subject, by administering to said subject an effective amount of a 11β-HSD1 reductase inhibitor, wherein the inhibitor is a nucleic acid.

In yet another embodiment, the invention pertains, at least in part, to a method for treating a glucocorticoid associated state in a subject, by administering to the subject an effective amount of a 11β-HSD1 reductase inhibitor-in combination with an effective amount of a 17α-hydroxylase inhibitor, a 17-HSD inhibitor, 20α-reductase inhibitor, or a 20β-reductase inhibitor.

In another embodiment, the invention pertains, at least in part, to a method for increasing the concentration of glucocorticoids in a tissue of a subject. The method includes administering to the subject an effective amount of a 11β-HSD1 dehydrogenase inhibitor, such as, for example, 3α,5β-reduced-11β-OH-testosterone, 3α, 5α-reduced-11β-OH-progesterone, 3α, 5α-reduced-11β-OH-testosterone, 3α, 5α-reduced-11β-OH-androstendione, 3α, 5α-reduced-corticosterone, 3α, 5α-reduced-aldosterone, 3α, 5α-reduced-progesterone, 3α, 5α-reduced testosterone, 3α, 5α-reduced-chenodeoxycholic acid, 3α,5β-reduced deoxycorticosterone, 3α,5β-reduced-chenodeoxycholic acid, 3α, 5β-reduced progesterone, 3α,5β-reduced testosterone, 3α, 5α-reduced deoxycorticosterone, or 11μ-OH testosterone. In another embodiment, the 11β-dehydrogenase inhibitor is a nucleic acid.

In yet another embodiment, the invention pertains, at least in part, to a method for increasing the concentration of glucocorticoids in a tissue of a subject. The method includes administering to the subject an effective amount of a 11β-HSD1 dehydrogenase inhibitor in combination with an effective amount of a 17α-hydroxylase inhibitor, a 17-HSD inhibitor, a 20α-reductase inhibitor or a 20β-reductase inhibitor.

In yet another embodiment, the invention pertains, at least in part, to a method for increasing the concentration of glucocorticoids in a tissue of a subject, comprising administering to a subject an effective amount of a 11β-HSD1 dehydrogenase inhibitor, such that the concentration of glucocorticoids in said tissue are increased, wherein said 11β-HSD1 dehydrogenase inhibitor is 3α, 5α-reduced-11β-OH-progesterone, 3α, 5α-reduced-11β-OH-testosterone, 3α, 5α-reduced-11I I-OH-androstendione, 3α, 5α-reduced-11β-OH-pregnenolone, 3α, 5α-reduced-11β-OH-dehydro-epiandrostenedione, 3α, 5α-reduced-corticosterone, 3α, 5α-reduced-aldosterone, 3α, 5α-reduced-pregnenolone, 3α, 5α-reduced-dehydro-epiandrostenedione, 11β-OH progesterone, 11β-OH testosterone, 11β-OH-pregnenolone, 11β-OH-dehydro-epiandrostenedione, 3α, 5α-reduced-progesterone, 3α, 5α-reduced testosterone, 3α, 5α-reduced-chenodeoxycholic acid, 3α, 5β-reduced deoxycorticosterone, 3α, 5β-reduced-chenodeoxycholic acid, 3α, 5β-reduced progesterone, 3α, 5β-reduced testosterone, or a pharmaceutically acceptable prodrug or salt thereof. In another embodiment, the 11β-HSD1 dehydrogenase inhibitor is a nucleic acid.

In yet another embodiment, the invention also pertains, at least in part, to a method for treating hypertension in a subject, by administering to the subject an effective amount of a 11β-HSD1 reductase inhibitor, such as, for example, 11-keto-progesterone, 11-keto-testosterone, 11-keto-androsterone, 11-keto-pregnenolone, 11-keto-dehydro-epiandrostenedione, 3α, 5α-reduced-11-keto-progesterone, 3α, 5α-reduced-11-keto-testosterone, 3α, 5α-reduced-11-keto-androstenedione, 3α, 5α-tetrahydro-11-dehydro-corticosterone, 3α, 5α-reduced-11-keto-pregnenolone, 3α-5β-reduced deoxycorticosterone, 3α, 5α-reduced deoxycorticosterone, 3α,5α-reduced progesterone, 3α, 5α-reduced testosterone or 3α, 5α-reduced-11-keto-dehydro-epiandrostenedione.

In yet another embodiment, the invention also pertains, at least in part, to a method for treating hypertension in a subject, by administering to the subject an effective amount of a 11β-HSD1 reductase inhibitor, such as, for example, a nucleic acid.

In an alternate embodiment, the invention pertains, at least in part, to a method for treating hypertension in a subject, by administering to the subject an effective amount of a 11β-HSD1 reductase inhibitor in combination with an effective amount of a 17α-hydroxylase inhibitor, a 17-HSD inhibitor, a 20α-reductase inhibitor or a 20β-reductase inhibitor.

In yet another embodiment, the invention pertains, at least in part, to a method for increasing insulin sensitivity of a tissue in a subject. The method includes administering an effective amount of a 11β-HSD1 reductase inhibitor to the subject. Examples of the 11β-HSD1 reductase inhibitor include nucleic acids, 11-keto-progesterone, 11-keto-testosterone, 11-keto-androsterone, 11-keto-pregnenolone, 11-keto-dehydro-epiandrostenedione, 3α, 5α-reduced-11-ketoprogesterone, 3α, 5α-reduced-11-keto-testosterone, 3α, 5α-reduced-11-keto-androstenedione, 3α,5α-tetrahydro-11-dehydro-corticosterone, 3α, 5α-reduced-11-keto-pregnenolone, 3α-5β-reduced deoxycorticosterone, 3α, 5α-reduced deoxycorticosterone, 3α,5α-reduced progesterone, 3α, 5α-reduced testosterone or 3α, 5α-reduced-11-keto-dehydro-epiandrostenedione.

In another embodiment, the invention pertains, at least in part, to a pharmaceutical composition comprising an effective amount of 11β-OH-progesterone, 11β-OH-testosterone, 3α, 5β-reduced-11β-OH-progesterone, 3α,5β-reduced-11β-OH-testosterone, chenodeoxycholic acid, 3α, 5β-reduced-pregnenolone, 3α, 5β-reduced-dehydro-epiandrostenedione, 3α,5α-reduced-11β-OH-progesterone, 3α,5α-reduced-11β-OH-testosterone, 3α,5α-reduced-11β-OH-androstenedione, 11-keto-progesterone, 11-keto-testosterone, 11-keto-androstenedione, 3α,5α-reduced-11-keto-progesterone, 3α,5α-reduced-11-keto-testosterone, 3α, 5α-reduced-11β-OH-pregnenolone, 3α, 5α-reduced-11β-OH-dehydro-epiandrostenedione, 11β-OH-pregnenolone, 11β-OH-dehydro-epiandrostenedione, 3α, 5α-reduced-pregnenolone, 3α, 5α-reduced-dehydro-epiandrostenedione, 3α,5α-reduced-11-keto-androstenedione, 3α,5α-tetrahydro-11-dehydro-corticosterone, 3α,5α-reduced-corticosterone, 5α-dihydro-corticosterone, 3α, 5α-reduced aldosterone, 3α-5β-reduced deoxycorticosterone, 3α, 5α-reduced deoxycorticosterone, 3α,5α-reduced progesterone, 3α,5β-reduced deoxycorticosterone, 3α,5β-reduced-chenodeoxycholic acid, 3α, 5β-reduced progesterone, 3α, 5β-reduced testosterone, 3α, 5α-reduced deoxycorticosterone, 3α, 5α-reduced testosterone or pharmaceutically acceptable salts thereof, in combination with a 17α-hydroxylase inhibitor, a 17-hydroxy steroid dehydrogenase (17-HSD), a 20α-reductase inhibitor, or a 20β-reductase inhibitor.

In another embodiment, the invention pertains to a composition comprising a 11β-HSD1 reductase inhibitor, wherein said 11β-HSD1 reductase inhibitor is an siRNA.

In another embodiment, the invention pertains to a composition comprising an 11β-HSD2 dehydrogenase inhibitor, wherein said 11β-HSD2 dehydrogenase inhibitor is an siRNA.

In yet another embodiment, the invention pertains to methods for treating apparent adrenal insufficiency in a subject. The methods include comprising administering to the subject an effective amount of an 11β-HDSD1 dehydrogenase inhibitor or a 11β-HSD2 dehydrogenase inhibitor.

In yet another embodiment, the invention includes a method for increasing the half-life of glucocorticoid drugs in a subject. The method includes administering to the subject an effective amount of a 11β-HSD2 dehydrogenase inhibitor in combination with a glucocorticoid drug, such that the half life of the glucocorticoid drug in the subject is increased.

### BRIEF DESCRIPTION OF THE DRAWINGS

FIG. 1 is a bar graph which shows that the exposure of rat aortic rings to corticosterone and 11β-HSD2 antisense resulted in a statistically significant increase in the contractile response to phenylephrine.

FIG. 2 is a bar graph which shows that in aortic rings treated with 11β-HSD1 antisense, the contractile responses to all concentrations of phenylephrine were significantly increased compared to aortic rings treated with corticosterone and nonsense oligomers.

FIG. 3 is a bar graph which illustrates that 11-dehydro-corticosterone amplifies the contractile responses to phenylephrine in rat aortic rings.

FIG. 4 is a bar graph which shows that the conversion of corticosterone to 11-dehydrocorticosterone was lower than in aortic rings incubated with corticosterone and 11β-HSD1 nonsense oligomers.

FIGS. 5A-5D are representative HPLC chromatograms showing the metabolism of ³H-11-dehydrocorticosterone (11-dehydroB) by rat aortic rings. In FIGS. 5A and 5B, the analysis of the tissue is shown for 11β-HSD1 nonsense and 11β-HSD1 antisense, respectively. In FIGS. 5C and 5D, the analysis of the incubation media is shown for 11β-HSD1 nonsense and 11β-HSD1 antisense, respectively.

### DETAILED DESCRIPTION OF THE INVENTION

I. Glucocorticoids And 11β-HSD1 Reductase, 11β-HSD1 Dehydrogenase And 11β-HSD2 Dehydrogenase

Glucocorticoids can affect vascular tone by modifying the actions of several vasoactive substances. Glucocorticoids amplify the vasoconstrictive actions of adrenergic catecholamines and angiotensin II on vascular smooth muscle cells. It has been reported that glucocorticoids decrease the biosynthesis of both nitric oxide and prostaglandin I, and attenuate the vasorelaxant actions of atrial natriuretic peptide in vascular tissue. Thus, the multiple effects of glucocorticoids in vascular tissue operate to increase vascular tone. Since vascular smooth muscle cells contain both glucocorticoid and mineralocorticoid receptors it is possible that glucocorticoids mediate their effects in vascular tissue via either or both of these receptor types.

Glucocorticoids are metabolized in vascular and other tissue by two isoforms of 11β-hydroxysteroid dehydrogenase (11β-HSD). 11β-HSD2 is unidirectional and metabolizes glucocorticoids to their respective inactive 11-dehydro derivatives, using NAD⁺ as a co-factor. 11β-HSD1 is bi-directional and possesses both dehydrogenase activity as well as reductase activity. The reductase activity of 11β-HSD1 regenerates active glucocorticoids from the inactive 11-dehydro derivatives. 11β-HSD1 uses NADP⁺ as a co-factor. In vascular tissue, glucocorticoids amplify the pressor responses to catecholamines and angiotensin II and down-regulate certain depressor systems such as nitric oxide and prostaglandins. Both 11β-HSD2 and 11β-HSD1 are believed to regulate glucocorticoid levels in vascular tissue and are part of additional mechanisms that control vascular tone.

Glucocorticoids are known to play an important role in the regulation of vascular tone and blood pressure. Glucocorticoid receptors and mineralocorticoid receptors are present in aorta, mesenteric arteries and rat vascular smooth muscle cells in culture. Glucocorticoids can bind to and activate glucocorticoid receptors (and possibly mineralocorticoid receptors) to potentiate the vasoconstrictive effects of both catecholamines and Ang II. Human and rat vascular endothelial cells contain both 11β-HSD2 and 11β-HSD1. It is generally understood that 11β-HSD2 operates to protect both mineralocorticoid receptors and glucocorticoid receptors from excessive stimulation by glucocorticoids. It has been noted that glucocorticoids further amplify the contractile effects of phenylephrine and Ang II when 11β-HSD enzyme activity is inhibited.

Rat vascular smooth muscle cells contain only 11β-HSD1. Under “physiologic conditions,” 11β-HSD1 acts largely as a reductase generating active corticosterone from inactive 11-dehydro-corticosterone.

11β-HSD1 reductase has an important role as a generator of active glucocorticoids in vascular tissue. 11β-HSD inactivates glucocorticoid molecules, allowing lower circulating levels of aldosterone to maintain renal homeostasis. Human and rat vascular endolethial cells contain both 11β-HSD1 and 11β-HSD2.

11β-HSD2 operates to protect both mineralocorticoid receptors and glucocorticoid receptors from excessive stimulation by glucocorticoids. It has also been shown that glucocorticoids further amplify the contractile effects of phenylephrine (PE) and Ang II when 11β-HSD1 or 2 dehydrogenase enzyme activity is inhibited.

II. Methods of Treating Glucocorticoid Associated States

In an embodiment, the invention pertains, at least in part, to a method for treating a glucocorticoid associated state in a subject. The method includes administering to the subject an effective amount of a 11β-HSD1 reductase modulating compound, such that the subject is treated.

The term “glucocorticoid associated states” include states which are associated with the presence or absence of aberrant amounts of glucocorticoids, particularly local levels in target tissues. It includes states which can be treated by modulating, e.g., inhibiting, the activating of a 11β-HSD1 reductase, or, alternatively, 11β-HSD1 dehydrogenase or 11β-HSD2 dehydrogenase. The term includes 11β-HSD1 reductase associated states. Examples of glucocorticoid associated states include blood pressure disorders, obesity, diabetes mellitus, interocular pressure, lung disorders, and neurological disorders. The glucocorticoid associated states may also include states associated with undesirable levels of glucocorticoids in adipose tissue, epithelial tissue in the eye, and interocular pressure.

“11β-HSD1 reductase associated state” includes states which can be treated by the administration of an 11β-HSD1 reductase modulating compound, e.g., an 11β-HSD1 reductase inhibitor. In certain embodiments, these states may be characterized by undesirable amounts of glucocorticoids in a tissue, fluid, or elsewhere in the subject.

The term “blood pressure disorders” include disorders which are associated with or characterized by abnormal or undesirable blood pressure. Examples of blood pressure disorders include, but are not limited to, high blood pressure, congestive heart failure, chronic heart failure, left ventricular hypertrophy, acute heart failure, myocardial infarction, cardiomyopathy, hypotension, hyponatremia, and hypertension, e.g., arterial hypertension and pulmonary hypertension.

The term “lung disorders” include disorders caused by or related to the presence or absence of glucocorticoids which can be treated by the compounds of the invention, for example, 11β-HSD1 reductase inhibitors. The lung contains considerable 11β-HSD1 activity (Nicholas and Lugg, J Steroid Biochem 17:113-118, 1982). During fetal development, there is little reductase activity but enzymatic activity increases significantly during lung maturation following birth. In circumstances where excess glucocorticoids are present in lung, there is a predisposition to pulmonary hypertension with an increase in pulmonary artery wall thickness (Cras et al. Am J Physiol Lung Cell Mol Physiol 278:L822-829, 2000) and collagen accumulation (Poiani et al Am J Respir Crit Care Med 149:994-999, 1994). Moreover glucocorticoids enhance endothelin receptor expression in lung (Shima J Pediatr Surg 35:203-207, 2000), a factor contributing to increased vascular resistance in the pulmonary arteries.

Another example of a glucocorticoid associated state is insulin insensitivity. High concentrations of cortisol in the liver substantially reduce insulin sensitivity, which increases gluconeogenesis and raises blood sugar levels of a subject. This effect is particularly disadvantageous in subjects suffering from impaired glucose tolerance or diabetes mellitus. In Cushing's syndrome, the antagonism of insulin can provoke diabetes mellitus in subjects. The 11β-HSD1 reductase inhibitors can be used to inhibit hepatic gluconeogenesis.

Another example of a glucocorticoid associated state is obesity (including centripetal obesity). It is thought that inhibition of the 11β-HSD1 reductase may reduce the effects of insulin resistance in adipose tissue in subjects. Not to be limited by theory, but it is thought that by decreasing insulin resistance will result in greater tissue utilization of glucose and fatty acids, thus reducing circulating levels.

Another example of a glucocorticoid associated state are neurological disorders. Glucocorticoid excess potentiates the action of certain neurotoxins, which leads to neuronal dysfumction and loss. Examples of neurological disorders that may be treated by include neuronal dysfunction and loss due to, for example, glucocorticoid potentiated neurotoxicity. Glucocorticoids may be involved in the cognitive impairment of aging with or without neuronal loss and also in dendritic attenuation. Furthermore, glucocorticoids have been implicated in the neuronal dysfunction of major depression.

Other examples of neurological disorders which may be treatable using the 11β-HSD1 reductase, 11β-HSD1 dehydrogenase, or 11β-HSD2 dehydrogenase modulators, e.g., inhibitors, of the invention, include both neuropsychiatric and neurodegenerative disorders such as Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amylotropic lateral sclerosis (ALS), progressive supranuclear palsy, epilepsy, and Creutzfeldt-Jakob disease; autonomic function disorders such as hypertension and sleep disorders, and neuropsychiatric disorders, such as depression, schizophrenia, schizoaffective disorder, Korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), bipolar affective neurological disorders, e.g., migraine and obesity, cognitive impairment of old age, and traumatic brain injury.

Another example of a glucocorticoid associated state is disorders characterized, by, for example, apparent adrenal insufficiency. Examples of such disorders and states include surgery, post-surgery, sepsis, shock, hypotension, hyponatremia, and conditions where it would be beneficial for a subject for increased glucocorticoid levels in plasma and tissues.

The term “subject” includes subjects capable of suffering from a glucocorticoid associated states, such as mammals. Examples of mammals include dogs, cats, bears, rabbits, mice, rats, goats, cows, sheep, horses, and, preferably, humans. The subject may be suffering from or at risk of suffering from a glucocorticoid associated state, e.g., a blood pressure associated disorder (e.g., hypertension, ocular hypertension, etc.), obesity, diabetes, a neurological disorder, or apparent adrenal insufficiency. The subject may be undergoing surgery or treatment for sepsis, hypotension, hyponatremia, or shock.

The term “treat” or “treating” includes the prevention, alleviation or reduction of at least one symptom or other indication of a particular glucocorticoid associated state. In one embodiment, the associated state is a blood pressure associated disorder, e.g., hypertension, and the administration of the modulating compound modulates, e.g., reduces, the blood pressure of the subject.

The term “effective amount” of the 11β-HSD1 reductase, 11β-HSD1 dehydrogenase, or 11β-HSD2 dehydrogenase modulating compound is that amount necessary or sufficient to treat or prevent a particular glucocorticoid associated state, e.g. prevent the various morphological and somatic symptoms of a glucocorticoid associated state. The effective amount can vary depending on such factors as the size and weight of the subject, the type of illness, or the particular 11β-HSD1 reductase, 11β-HSD1 dehydrogenase, or 11β-HSD2 dehydrogenase modulating compound, e.g., inhibiting, compound.

In a further embodiment, the 11β-HSD1 reductase, 11β-HSD1 dehydrogenase, or 11β-HSD2 dehydrogenase modulating compound may be administered in combination with a pharmaceutically acceptable carrier.

In a further embodiment, the invention pertains to a method for treating a blood pressure associated disorder, e.g., hypertension, in a subject, by administering to the subject an effective amount of an 111β-HSD1 reductase, 11β-HSD1 dehydrogenase, or 11β-HSD2 dehydrogenase modulating, e.g., inhibiting, compound.

In another embodiment, the invention features a method for decreasing the concentration (or amount) of glucocorticoids in a tissue of a subject. The method includes administering an effective amount of a selective 11β-HSD1 reductase inhibitor, such that the concentration of glucocorticoids in the tissue are decreased. In a further embodiment, the 11β-HSD1 reductase inhibitor is a small molecule, e.g., a steroid or a derivative thereof.

Examples of tissues where the concentration of glucocorticoids in a subject may be decreased include tissues which express 11β-HSD1 or other wise contain an undesirable concentration of glucocorticoids. Examples of such tissues include a subject's blood, liver, eye, lung, muscle, adipose tissue, nerve tissue, brain, or vascular tissue.

In another embodiment, the invention features a method for treating a blood pressure associated disorder, such as, for example, hypertension, in a subject. The method includes administering to a subject an effective amount of a 11β-HSD1 reductase inhibitor, such that the subject is treated. In a further embodiment, the 11β-HSD1 reductase inhibitor is a selective inhibitor. In another embodiment, the reductase inhibitor is a small molecule, e.g., a steroid or a derivative thereof.

In another embodiment, the invention features a method for increasing insulin sensitivity of a tissue in a subject. The method includes administering to a subject an effective amount of a selective 11β-HSD1 reductase inhibitor, such that the insulin sensitivity of the tissue in the subject is increased. Examples of tissue where increased insulin sensitivity may be desirable include, for example, the subject's liver, muscle, nerve or adipose tissue.

In yet another embodiment, the invention features a method for increasing the concentration of glucocorticoids in a tissue of a subject. The method includes administering to a subject an effective amount of a selective 11β-HSD1 dehydrogenase inhibitor, such that the concentration of glucocorticoids in the tissue are increased.

The tissue may be any tissue which an increase in the concentration of glucocorticosteroids is desired. Examples of such tissues include, but are not limited to, subject's liver, blood, lung, eye, muscle, adipose tissue, nerve tissue, brain, and vascular tissue.

In another embodiment, the invention features a method for increasing the concentration of glucocorticoids in a tissue of a subject. The method includes administering to a subject an effective amount of a selective 11β-HSD2 dehydrogenase inhibitor, such that the concentration of glucocorticoids in the tissue are increased.

The tissue may be any tissue which an increase in the concentration of glucocorticoids is desired. Examples of such tissues include, but are not limited to, subject's liver, eye, blood, lung, muscle, adipose tissue, nerve tissue, brain, kidney, and vascular tissue.

The invention also includes a method for selectively inhibiting 11β-HSD1 reductase. The method includes contacting 11β-HSD1 reductase with a selective 11β-HSD1 reductase inhibitor.

In yet another embodiment, the invention includes a method for selectively inhibiting 11β-HSD1 dehydrogenase. The method includes contacting 11β-HSD1 dehydrogenase with a selective 11β-HSD1 dehydrogenase inhibitor.

In another embodiment, the invention pertains to a method for treating apparent adrenal insufficiency in a subject, by administering to the subject an effective amount of an 11β-HDSD1 dehydrogenase inhibitor or a 11β-HSD2 dehydrogenase inhibitor. In a further embodiment, the subject is undergoing, about to undergo, or has undergone surgery. The subject also may be suffering from or at risk of suffering from sepsis, hyponatremia or hypotension. The 11β-HSD1 or 11β-HSD2 inhibitors may be selective inhibitors.

In certain embodiments, the 11β-HSD1 dehydrogenase inhibitor is administered in combination with an 11β-HSD2 dehydrogenase inhibitor to the subject.

The language “in combination with” a second inhibitor includes co-administration of the first inhibitor with the second agent, administration of the first inhibitor first, followed by the second inhibitor and administration of the second inhibitor first, followed by the first inhibitor.

The invention also includes a method for increasing the half-life of glucocorticoid drugs in a subject. The method includes administering to a subject an effective amount of a 11β-HSD2 dehydrogenase inhibitor in combination with said glucocorticoid drug.

The term “half life” includes the length of time the drug is retained in the body in its active form. In a further embodiment, the half-life of the particular drug is increased 10% or greater, 20% or greater, 30% or greater, 50% or greater, 100% or greater, 150% or greater, or 200% or greater.

The term “glucocorticoid drug” include drugs such as 11-keto glucocorticoid drugs and other drugs which may be metabolized to cortisol by the kidney. Examples of 11-keto glucocorticoid drugs include prednisone, 9α-fluorocortisone, 9α-fluoro-16α-hydroxyprednisone, and dexamethasone.

III. 11β-HSD1 Reductase Modulating Compounds, 11β-HSD1-Dehydrogenase Modulating Compounds And 11β-HSD2 Dehydrogenase Modulating Compounds

The term “11β-HSD1 reductase modulating compound” include compounds and agents (e.g., oligomers, proteins, etc.) which modulate or inhibit the activity of 11β-HSD1 reductase. In an advantageous embodiment, the 11β-HSD1 reductase modulating compound is an 11β-HSD1 reductase inhibitor (also referred to as “11β-HSD1 reductase inhibiting compound”). The 11β-HSD1 reductase modulating compound may be a small molecule, e.g., a compound with a molecular weight below 10,000 daltons.

In a further embodiment, the 11β-HSD1 reductase modulating compound is a selective inhibitor of 11β-HSD1 reductase. The term “selective 11β-HSD1 reductase inhibitor” includes compounds which selectively inhibit the reductase activity of 11β-HSD1 as compared to the dehydrogenase activity. In a further embodiment, the reductase activity is inhibited at a rate about 2 times or greater, about 3 times or greater, about 4 times or greater, about 5 times or greater, about 10 times or greater, about 15 times or greater, about 20 times or greater, about 25 times or greater, about 50 times or greater, about 75 times or greater, about 100 times or greater, about 150 times or greater, about 200 times or greater, about 300 times or greater, about 400 times or greater, about 500 times or greater, about 1×10³ times or greater, about 1×10⁴ times or greater, about 1×10⁵ times or greater, or about 1×10⁶ or greater as compared with the inhibition of the dehydrogenase activity of 11β-HSD1.

In a further embodiment, the 11β-HSD1 reductase modulating compound may be a steroid or a steroid derivative. The steroid ring system is generally numbered according to IUPAC conventions, as shown below:

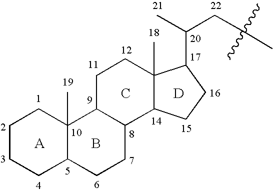

Examples of 11β-HSD1 reductase modulating compounds include 11-keto steroid compounds, e.g., compounds with the steroid ring system with a carbonyl functional group at the 11-position of the steroid ring. Examples of steroid compounds with an 11-keto group include, for example, 11-keto progesterone, 11-keto-testosterone, 11-keto-androsterone, 11-keto-pregnenolone, 11-keto-dehydro-epiandrostenedione, 3α, 5α-reduced-11-ketoprogesterone, 3α, 5α-reduced-11-keto-testosterone, 3α, 5α-reduced-11-keto-androstenedione, 3α,5α-tetrahydro-11-dehydro-corticosterone, 3α, 5α-reduced-11-keto-pregnenolone, and 3α, 5α-reduced-11-keto-dehydro-epiandrostenedione. Other examples of 11β-HSD1 reductase modulating compounds of the invention are compounds which conserve a least a portion of the steroid nucleus. These compounds may have additional substituents, such as fatty acid tails at the 22 position, or other modifications (e.g., substitutions of the ring by halogens, formation of esters or other protecting groups for the hydroxyl groups of the steroids, or replacement of functional groups with others that may, for example, advantageously, lengthen the time the molecule is in its active form in a subjects body. Alternatively, the modifications can be such that the reduce the time the compound is in its active form in a subject's body.

Examples of 11β-HSD1 reductase modulating compounds also include 3α, 5α-reduced steroid compounds. Examples of 3α, 5α-reduced steroid compounds include 3α, 5α-reduced-11-ketoprogesterone, 3α, 5α-reduced-11-keto-testosterone, 3α, 5α-reduced-11-keto-androstenedione, 3α,5α-tetrahydro-11-dehydro-corticosterone, 3α, 5α-reduced-11-keto-pregnenolone, 3α, 5α-reduced corticosterone, 3α,5α-reduced progesterone, 3α, 5α-reduced testosterone and 3α, 5α-reduced-11-keto-dehydro-epiandrostenedione.

In a further embodiment, the 11β-HSD1 reductase modulating compound is 3α, 5β reduced, e.g., 3α, 5β-reduced deoxycorticosterone.

Steroid derivatives include compounds with a steroid ring structure optionally substituted with additional substituents which allow the compound to perform its intended function. It should be noted that the steroid compounds may be converted to the active form of the modulating compound within the subject. The invention includes administering compounds which are in other forms, e.g., prodrugs, and which are metabolized in vivo to yield the 11β-HSD1 reductase modulating compounds described herein.

In one embodiment, the 11β-HSD1 reductase inhibitors possess IC₅₀'s less than about 0.5 μM using 600 nanoM 11-dehydro-corticosterone substrate concentration and testicular leydig cell homogenates. Methods for testing the IC₅₀'s of the enzymes are described in further detail in Latif, S. A. et al. Steroids 62: 230-237, 1997. In another embodiment, the 11β-HSD1 reductase inhibitors have an IC₅₀ of 80 μM or less, or, preferably, 15 μM or less. In another embodiment, the 11β-HSD1 reductase inhibitors have an IC₅₀ of less than 100 μM.

Other examples of 11β-HSD1 reductase modulating compounds include carbenoxolone and derivatives thereof. In other embodiments, 11β-HSD1 reductase modulating compound is a nucleic acid. In another embodiment, the 11β-HSD1 reductase inhibitor is an antisense nucleic acid. In another embodiment, the 11β-HSD1 reductase inhibitor is a siRNA.

The basic mechanism of RNA interference can be understood as a two step process (Zamore P. D., Nature Struc. Biol., 8, 9, 746-750, (2001)). First, the dsRNA is cleaved to yield short interfering RNAs (siRNAs) of about 21-23 nt length with 5′ terminal phosphate and 3′ short overhangs (−2 nt). Then, the siRNAs target the corresponding mRNA sequence specific for destruction (Fire A. et al., Nature, Vol 391, (1998); Hamilton A J et al. Science, 286, 950-952, (1999); Zamore P D. et al. Cell, 101, 25-33, (2000); Elbashir S M. et al., Genes & Development, 15, 188-200, (2001); Bernstein E. et al. Nature 409, 363-366, 2001).

It has been demonstrated that chemically synthesized 21 nt siRNA duplexes specifically suppress expression of endogenous and heterologeous genes in different mammalian cell lines, including human kidney and HeLa cells (Elbashir S M. et al., Nature, 411, 494-498, (2001)). It was discovered that no unspecific effects occurred in mammalian cells by transfection of short sequences (<30 nt). It was suggested that 21 nt siRNA duplexes provide a new tool for studying gene function in mammalian cells and may eventually be used as gene-specific therapeutics.

It was also found that siRNAs mediated RNAi in cell extracts and synthetic siRNAs can induce gene-specific inhibition of expression in C. elegans and in cell lines from humans and mice (Caplen, N. J. et al. PNAS 171251798, 1-6, (2001)30). It was also shown that siRNAs can have direct effects on gene expression in C. elegans and mammalian cell culture in vivo.

Methods for making and using siRNAs are described in, for example, WO 01/75164, US 2002/0137210, WO 01/29058, WO 02/072762, WO 02/059300, WO 02/44321, WO 01/92513, WO 01/68836, US 2002/0173478, US 2002/0160393, US 2002/0162126, US 2002/0137709, US 2002/0132788, US 2002/0086356, and WO 99/32619; each of which is expressly incorporated herein by reference.

In one embodiment, the 11β-HSD1 reductase inhibitor is a double stranded RNA oligomer, wherein the antisense strand is complementary to at least a portion of SEQ. ID. No. 1. In one embodiment, the portion is 40 base pairs or less, 35 base pairs or less, 30 base pairs or less, 29 base pairs or less, 28 base pairs or less, 27 base pairs or less, 26 base pairs or less, 25 base pairs or less, 24 base pairs or less, 23 base pairs or less, 22 base pairs or less, 21 base pairs or less, 20 base pairs or less, 19 base pairs or less, or about 18 base pairs or less. In another embodiment, the oligomer has 10 or more base pairs, 11 or more base pairs, 12 or more base pairs, 13 or more base pairs, 14 base pairs or more, 15 base pairs or more, 16 base pairs or more, 17 base pairs or more, 18 base pairs or more, or 19 base pairs or more. In another embodiment, the 11β-HSD1 reductase inhibitor has an antisense strand having the sequence 5′-CAT AAC TGC CGT CCA ACA GC-3′ (SEQ ID NO. 2).

The term “11β-HSD1 dehydrogenase modulating compound” include compounds and agents (e.g., oligomers, proteins, etc.) which modulate or inhibit the activity of 11β-HSD1 dehydrogenase. In an advantageous embodiment, the 11β-HSD1 dehydrogenase modulating compound is an 11β-HSD1 dehydrogenase inhibitor (also referred to as “11β-HSD1 dehydrogenase inhibiting compound”). The 11β-HSD1 dehydrogenase modulating compound may be a small molecule, e.g., a compound with a molecular weight below 10,000 daltons.

In a further embodiment, the 11β-HSD1 dehydrogenase modulating compound is a selective inhibitor of 11β-HSD1 dehydrogenase. The term “selective 11-HSD1 dehydrogenase inhibitor” includes compounds which selectively inhibit the dehydrogenase activity of 11β-HSD1 as compared to the reductase activity of 11β-HSD1. In a further embodiment, the dehydrogenase activity is inhibited at a rate about 2 times or greater, about 3 times or greater, about 4 times or greater, about 5 times or greater, about 10 times or greater, about 15 times or greater, about 20 times or greater, about 25 times or greater, about 50 times or greater, about 75 times or greater, about 100 times or greater, about 150 times or greater, about 200 times or greater, about 300 times or greater, about 400 times or greater, about 500 times or greater, about 1×10³ times or greater, about 1×10⁴ times or greater, about 1×10⁵ times or greater, or about 1×10⁶ or greater as compared with the inhibition of the reductase activity of 11β-HSD1.

In one embodiment, the 11β-HSD1 dehydrogenase inhibitor is a small molecule, such as a steroid or a derivative thereof. In a further embodiment, the steroid is 3α, 5β-reduced. Examples of 3α,5β-reduced steroids include 3α, 5β-reduced-11β-OH-progesterone, 3α, 5β-reduced-11β-OH-testosterone, chenodeoxycholic acid, 3α, 5β-reduced-pregnenolone, 3α, 5β-reduced-dehydro-epiandrostenedione, 3α, 5β-reduced-progesterone, 3α, 5β-reduced deoxycorticosterone, 3α, 5β-reduced-chenodeoxycholic acid, 3α, 5β-reduced progesterone, 3α, 5β-reduced testosterone, 3α, 5β-reduced chenodoxycholic acid, 3α, 5β-testosterone, and deoxy-corticosterone.

In another embodiment, the 11β-HSD1 dehydrogenase inhibitor is a 3α, 5α-reduced steroid. Examples of such steroids include 3α, 5α-reduced-11β-OH-progesterone, 3α, 5β-reduced-11β-OH-testosterone, 3α, 5α-reduced-11β-OH-androstendione, 3α, 5α-reduced-11β-OH-pregnenolone, 3α, 5α-reduced-11β-OH-dehydro-epiandrostenedione, 3α, 5α-reduced-corticosterone, 3α, 5α-reduced-aldosterone, 3α, 5α-reduced-pregnenolone, 3α, 5α-reduced-progesterone, 3α, 5α-reduced testosterone, 3α, 5α-deoxycorticosterone, and 3α, 5α-reduced-chenodeoxycholic acid. Other examples of steroids which can be used as 11β-HSD1 dehydrogenase inhibitors include 11β-OH progesterone, 11β-OH testosterone, 11β-OH-pregnenolone, 11β-OH-dehydro-epiandrostenedione, glycyrrhetinic acid or carbenoxolone.

In one embodiment, the 11β-HSD1 dehydrogenase inhibitor has an IC₅₀ of 0.5 μM or less. In another embodiment, the 11β-HSD1 dehydrogenase inhibitor has an IC₅₀ of 100 μM or less, 80 μM or less, or 20 μM or less (using 100 nM corticosterone substrate concentration and testicular Leydig cell homogenates).

The term “11β-HSD2 dehydrogenase inhibitor” includes agents which inhibit or decrease the dehydrogenase activity of 11β-HSD2.

In one embodiment, the 11β-HSD2 dehydrogenase inhibitor is a small molecule, such as a steroid or a derivative thereof. In one embodiment, the steroid is 3α, 5α-reduced. Examples of 11β-HSD2 dehydrogenase inhibitors include, but are not limited to, 3α, 5α-reduced-11β-OH-progesterone, 3α, 5α-reduced-11β-OH-testosterone, 3α, 5α-reduced-11β-OH-androstenedione, 3α, 5α-reduced-11-keto-progesterone, 3α, 5α-reduced-11-dehydro-corticosterone, 3α, 5α-reduced-corticosterone, 3α, 5α-reduced-11β-OH-pregnenolone, 3α, 5α-reduced-11β-OH-dehydro-epiandrostenedione, 3α, 5α-reduced-pregnenolone, 3α, 5α-reduced-dehydro-epiandrostenedione, 3α, 5α-reduced aldosterone, and 3α, 5α-reduced deoxycorticosterone. Other examples of 11β-HSD2 dehydrogenase inhibitors include 11β-OH-progesterone, 11β-OH-pregnenolone, 11β-OH-dehydro-epiandrostenedione, 11β-OH-testosterone, 11-keto-progesterone, 5α-dihydro-corticosterone, 3α, 5α-reduced deoxy-corticosterone, glycyrrhetinic acid or carbenoxolone.

In other embodiments, 11β-HSD2 dehydrogenase modulating compound is a nucleic acid. In another embodiment, the 11β-HSD2 dehydrogenase inhibitor is an antisense nucleic acid. In another embodiment, the 11β-HSD2 dehydrogenase inhibitor is a siRNA.

In one embodiment, the 11β-HSD2 dehydrogenase inhibiting compounds have IC₅₀'s less than 2.5 μM (using 50 nM corticosterone substrate concentration and sheep kidney microsomes). In another embodiment, the 11β-HSD2 dehydrogenase inactive compounds have an IC₅₀ of less than 10 μM.

In one embodiment, the 11β-HSD2 dehydrogenase inhibitor is a double stranded RNA oligomer, wherein the antisense strand is complementary to at least a portion of SEQ. ID. No.3. In one embodiment, the portion is 40 base pairs or less, 35 base pairs or less, 30 base pairs or less, 29 base pairs or less, 28 base pairs or less, 27 base pairs or less, 26 base pairs or less, 25 base pairs or less, 24 base pairs or less, 23 base pairs or less, 22 base pairs or less, 21 base pairs or less, 20 base pairs or less, 19 base pairs or less, or about 18 base pairs or less. In another embodiment, the oligomer has 10 or more base pairs, 11 or more base pairs, 12 or more base pairs, 13 or more base pairs, 14 base pairs or more, 15 base pairs or more, 16 base pairs or more, 17 base pairs or more, 18 base pairs or more, or 19 base pairs or more. In another embodiment, the 11β-HSD2 dehydrogenase inhibitor has an antisense strand having the sequence 5′-AGG CCA GCG CTC CAT GAC TT-3′ (SEQ ID NO 4).

The invention also pertains to each of the nucleic acids described herein as well as pharmaceutical compositions comprising these nucleic acids.

Examples of 11β-HSD1-reductase, 11β-HSD1-dehydrogenase and 11β-HSD2 dehydrogenase modulating compounds are described in Table 1.

TABLE 1

| Compound | | 11β-HSD1 | 11β-HSD1 | 11β-HSD2 |
|---|---|---|---|---|
| Name | Structure | Reductase | Dehydrogenase | Dehydrogenase |
| 11β-OH- progesterone | 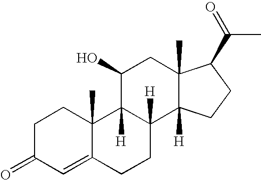 | No Inhibition | Potent Inhibitor (Non-Selective) | Potent Inhibitor (Non-Selective) |
| 11β-OH- testosterone | 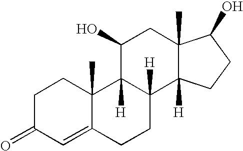 | No Inhibition | Inhibitor (Non-Selective) | Inhibitor (Non-Selective) |
| 3α,5β-reduced- 11β-OH- progesterone | 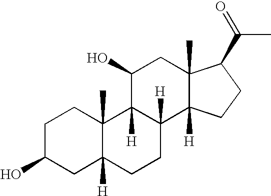 | No Inhibition | Moderate Inhibitor | No Inhibition |
| 3α,5β-reduced- 11β-OH- testosterone | 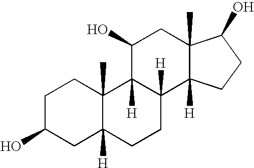 | No Inhibition | Moderate Inhibitor | No Inhibition |
| chenodeoxycholic acid (3α,5β-reduced steroid) | 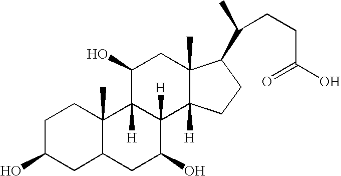 | No Inhibition | Selective inhibitor | No Inhibition |
| 3α,5α-reduced- 11β-OH- progesterone | 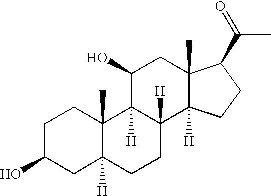 | No Inhibition | Potent Inhibitor (Non-Selective) | Potent Inhibitor (Non-Selective) |
| 3α,5α-reduced- 11β-OH- testosterone | 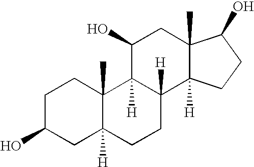 | No Inhibition | Potent Inhibitor (Non-Selective) | Potent Inhibitor (Non-Selective) |
| 3α,5α-reduced- 11β-OH- androstenedione | 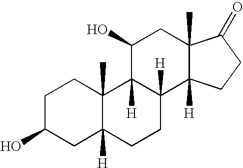 | No Inhibition | Moderate Inhibitor | Potent Inhibitor (Non-Selective) |
| 11-Keto- progesterone | 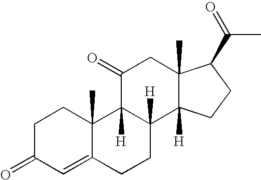 | Selective Inhibitor | No Inhibition | Potent Inhibitor |
| 11-Keto- testosterone | 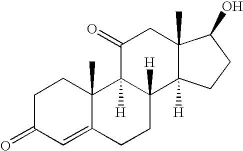 | Selective Inhibitor | No Inhibition | No Inhibition |
| 11-Keto- androstenedione | 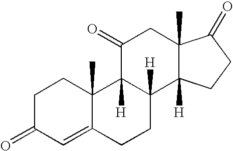 | Selective Inhibitor | No Inhibition | No Inhibition |
| 3α,5α-reduced- 11-keto- progesterone | 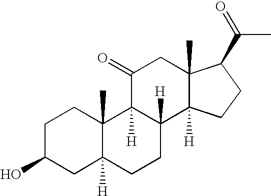 | Selective Inhibitor | No Inhibition | Potent Inhibitor |
| 3α,5α-reduced- 11-keto- testosterone | 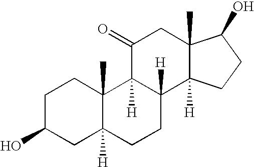 | Selective Inhibitor | No Inhibition | Not tested |
| 3α,5α-reduced- 11-keto- androstenedione | 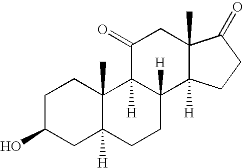 | Selective Inhibitor | No Inhibition | Not Tested |
| 3α,5α-tetrahydro- 11-dehydro- corticosterone | 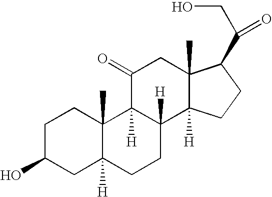 | Potent Inhibitor | No Inhibition | Potent Inhibitor |
| 3α,5α-reduced- corticosterone | 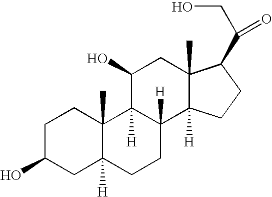 | No Inhibition | Potent Inhibitor | Potent Inhibitor |
| 5α-dihydro- corticosterone | 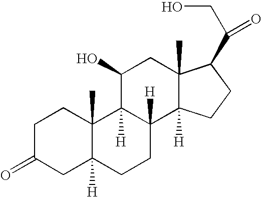 | No inhibition | Potent Inhibitor | Potent Inhibitor |
| 3α,5α-reduced aldosterone | 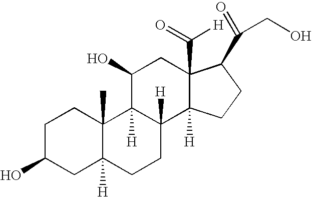 | No Inhibition | Moderate Inhibitor | Potent Inhibitor |

IV. 17α-Hydroxylase Inhibitors, 17-HSD Inhibitors 20α-Reductase Inhibitors And 20β-Reductase Inhibitors

The invention also pertains to administering to the subject a 17α-hydroxylase inhibitor, a 17-HSD inhibitor, a 20α-reductase inhibitor and/or a 20β-reductase inhibitor, in combination with the methods described above. The inhibitors can be any compound or substance known to inhibit any one of these enzymes. The 17α-hydroxylase, 17-HSD, 20α-reductase and/or 20β-reductase inhibitors are administered in combination with the compounds of the invention described herein.

The language “in combination with” another agent includes co-administration of the compound of the invention and the agent, administration of the compound of the invention first, followed by the other agent and administration of the other agent first, followed by the compound of the invention.

The 17α-hydroxylase, 17-HSD, 20α-reductase or 20β-reductase inhibitors can be found using assays for screening candidate or test compounds which bind to or modulate the activity of a 17α-hydroxylase, 17-HSD, 17-HSD, 20α-reductase or 20β-reductase protein or polypeptide or biologically active portion thereof. The sequences for 17α-hydroxylase is shown in SEQ ID No.5. The sequence for the 17-HSD is shown in SEQ ID No.6. The polypeptide sequence for 17-HSD is shown in SEQ ID. No.7. In another embodiment, the 17α-hydroxylase inhibitor, the 17-HSD inhibitor, the 20α-reductase, and/or the 20β reductase inhibitors are nucleic acid oligomers. In a further embodiment, the nucleic acid oligomers are siRNA and comprise at least a portion of SEQ ID No.5 or SEQ ID No. 6.

The test compounds can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1 994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. EngL. 33:2061; and in Gallop et al. (1994) J. Med Chem. 37:1233.

Libraries of compounds may be presented in solution (e.g, Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310).

Determining the ability of a 17α-hydroxylase, 17-HSD, 20α-reductase or 20β-reductase protein to bind to or interact with a target molecule (e.g., a steroid substrate) can be accomplished by determining direct binding. Determining the ability of the 17α-hydroxylase, 17-HSD, 20α-reductase or 20β-reductase protein to bind to or interact with a target molecule can be accomplished, for example, by coupling the 17α-hydroxylase, 17-HSD, 20α-reductase or 20β-reductase protein with a radioisotope or enzymatic label such that binding of the 17α-hydroxylase, 17-HSD, 20α-reductase or 20β-reductase protein to a target molecule can be determined by detecting the labeled 17α-hydroxylase, 17-HSD, 20α-reductase or 20β-reductase protein in a complex. For example, 17α-hydroxylase, 17-HSD, 20α-reductase or 20β-reductase proteins can be labeled with ¹²⁵I, ³⁵S, ¹⁴C, or ³H, either directly or indirectly, and the radioisotope detected by direct counting of radioemmission or by scintillation counting. Alternatively, 17α-hydroxylase, 17-HSD, 20α-reductase or 20β-reductase proteins can be enzymatically labeled with, for example, horseradish peroxidase, alkaline phosphatase, or luciferase, and the enzymatic label detected by determination of conversion of an appropriate substrate to product.

In yet another embodiment, an assay of the present invention is a cell-free assay in which a 17α-hydroxylase, 17-HSD, 20α-reductase or 20β-reductase protein or biologically active portion thereof is contacted with a test compound and the ability of the test compound to bind to the 17α-hydroxylase, 17-HSD, 20α-reductase or 20β-reductase protein or biologically active portion thereof is determined. Binding of the test compound to the 17α-hydroxylase, 17-HSD, 20α-reductase or 20β-reductase protein can be determined either directly or indirectly. The assay may include contacting the 17α-hydroxylase, 17-HSD, 20α-reductase or 20β-reductase protein or biologically active portion thereof with a known compound which binds 17α-hydroxylase, 17-HSD, 20α-reductase or 20β-reductase to form an assay mixture, contacting the assay mixture with a test compound, and determining the ability of the test compound to interact with a 17α-hydroxylase, 17-HSD, 20α-reductase or 20β-reductase protein, wherein determining the ability of the test compound to interact with a 17α-hydroxylase, 17-HSD, 20α-reductase or 20β-reductase protein comprises determining the ability of the test compound to preferentially bind to 17α-hydroxylase, 17-HSD, 20α-reductase or 20β-reductase or biologically active portion thereof as compared to the known compound.

In another embodiment, the assay is a cell-free assay in which a 17α-hydroxylase, 17-HSD, 20α-reductase or 20β-reductase protein or biologically active portion thereof is contacted with a test compound and the ability of the test compound to modulate (e.g., stimulate or inhibit) the activity of the 17α-hydroxylase, 17-HSD, 20α-reductase or 20β-reductase protein or biologically active portion thereof is determined. Determining the ability of the test compound to modulate the activity of a 17α-hydroxylase, 17-HSD, 20α-reductase or 20β-reductase protein can be accomplished, for example, by determining the ability of the 17α-hydroxylase, 17-HSD, 20α-reductase or 20β-reductase protein to bind to a target molecule. Determining the ability of the 17α-hydroxylase, 17-HSD, 20α-reductase or 20β-reductase protein to bind to a target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of a 17α-hydroxylase, 17-HSD, 20α-reductase or 20β-reductase protein can be accomplished by determining the ability of the 17α-hydroxylase, 17-HSD, 20α-reductase or 20β-reductase protein to further modulate the activity of a target molecule.

In yet another embodiment, the cell-free assay involves contacting a 17α-hydroxylase, 17-HSD, 20α-reductase or 20β-reductase protein or biologically active portion thereof with a known compound which binds the 17α-hydroxylase, 17-HSD, 20α-reductase or 20β-reductase protein to form an assay mixture, contacting the assay mixture with a test compound, and determining the ability of the test compound to interact with the 17α-hydroxylase, 17-HSD, 20α-reductase or 20β-reductase protein, wherein determining the ability of the test compound to interact with the 17α-hydroxylase, 17-HSD, 20α-reductase or 20β-reductase protein comprises determining the ability of the 17α-hydroxylase, 17-HSD, 20α-reductase or 20β-reductase protein to preferentially bind to or modulate the activity of a target molecule.

It may be desirable to immobilize either 17α-hydroxylase, 17-HSD, 20α-reductase or 20β-reductase or its target molecule to facilitate separation of complexed from uncomplexed forms of one or both of the proteins, as well as to accommodate automation of the assay. Binding of a test compound to a 17α-hydroxylase, 17-HSD, 20α-reductase or 20β-reductase protein, or interaction of a 17α-hydroxylase, 17-HSD, 20α-reductase or 20β-reductase protein with a target molecule in the presence and absence of a candidate compound, can be accomplished in any vessel suitable for containing the reactants. Examples of such vessels include microtitre plates, test tubes, and micro-centrifuge tubes.

In one embodiment, the invention pertains to the 17α-hydroxylase, 17-HSD, the 20α-reductase, and the 20β-reductase inhibiting compounds which are found using the above described methods.

V. Pharmaceutical Compositions

In yet another embodiment, the invention pertains to a pharmaceutical composition for the treatment of a glucocorticoid associated state. The composition includes an effective amount of an 11β-HSD1 reductase, 11β-HSD1 dehydrogenase, or 11β-HSD2 dehydrogenase modulating, e.g., inhibiting, compound and a pharmaceutically acceptable carrier. In a further embodiment, the glucocorticoid associated state is a blood pressure disorder. In another embodiment, the pharmaceutical compositions may also comprise an inhibitor of 17α-hydroxylase, 17-HSD, 20α-reductase or 20β-reductase.

In another embodiment, the invention pertains, at least in part, to a pharmaceutical composition comprising an effective amount of 11β-OH-progesterone, 11β-OH-testosterone, 3α,5β-reduced-11β-OH-progesterone, 3α,5α-reduced-11β-OH-testosterone, chenodeoxycholic acid, 3α, 5α-reduced-pregnenolone, 3α, 5β-reduced-dehydro-epiandrostenedione, 3α,5α-reduced-11β-OH-progesterone, 3α,5α-reduced-11β-OH-testosterone, 3α,5α-reduced-11β-OH-androstenedione, 11-keto-progesterone, 11-keto-testosterone, 11-keto-androstenedione, 3α,5α-reduced-11-keto-progesterone, 3α,5α-reduced-11-keto-testosterone, 3α, 5α-reduced-11β-OH-pregnenolone, 3α, 5α-reduced-11β-OH-dehydro-epiandrostenedione, 11β-OH-pregnenolone, 11β-OH-dehydro-epiandrostenedione, 3α, 5α-reduced-pregnenolone, 3α, 5α-reduced-dehydro-epiandrostenedione, 3α,5α-reduced-11-keto-androstenedione, 3α,5α-tetrahydro-11-dehydro-corticosterone, 3α,5α-reduced-corticosterone, 5α-dihydro-corticosterone, 3α,5β-reduced deoxycorticosterone, 3α, 5α-reduced deoxycorticosterone, 3α, 5α-reduced progesterone, 3α, 5α-reduced testosterone, 3α, 5β-reduced deoxycorticosterone, 3α,5β-reduced-chenodeoxycholic acid, 3α, 5α-reduced progesterone, 3α,5β-reduced testosterone, 3α, 5α-reduced deoxycorticosterone, 3α, 5α-reduced aldosterone, and pharmaceutically acceptable salts thereof, in combination with a 17α-hydroxylase inhibitor, a 20α-reductase inhibitor, or a 20β-reductase inhibitor.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, ct-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal, pulmonary and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Sprays also can be delivered by mechanical, electrical, or by other methods known in the art.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial, antiparasitic and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form may be accomplished by dissolving or suspending the drug in an oil vehicle. The compositions also may be formulated such that its elimination is retarded by methods known in the art.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration or administration via inhalation is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually. Other methods for administration include via inhalation.

The language: “directed to” includes methods of administration, such as injection, which allow for the higher concentration or active amount of the inhibitor or drug to be located in kidney after administration.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats a glucocorticoid associated state.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

As set out above, certain embodiments of the present compounds can contain a basic functional group, such as amino or alkylamino, and are, thus, capable of forming pharmaceutically acceptable salts with pharmaceutically acceptable acids. The term “pharmaceutically acceptable salts” is art recognized and includes relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Farm. SCI. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances includes relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “prodrug” includes compounds with moieties which can be metabolized in vivo to a hydroxyl group or other functional group and moieties which may advantageously remain in vivo. Preferably, the prodrugs moieties are metabolized in vivo. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides.

The invention also pertains to any one of the methods described supra further comprising administering to the subject a pharmaceutically acceptable carrier.

### EXEMPLIFICATION OF THE INVENTION

### Example 1

### Ability of Corticosterone And 11-Dehydro-Corticosterone To Amplify the Contractile Responses of Phenylephrine

Experimental

Male Sprague-Dawley (1 50-200 g) rats were anesthetized with pentobarbital (50 mg/kg IP), and a median sternotomy was performed followed by the rapid removal of the thoracic aorta. The adventitia was removed, but the endothelium was left intact. The aorta was cut into 2-3 mm rings and individual rings were placed into a single well of a twenty four well culture plate and incubated at 37° C. under 95% O₂-5% CO₂. Each well contained 1 mL of DMEM/F12 containing 1% fetal bovine serum, streptomycin (100 μg/ml), penicillin (100 units/ml) and amphotericin (0.25 μg/ml). Aortic rings were incubated for 24 hours prior to contractility measurements with the following combinations of steroids, and antisense/nonsense oligonucleotides (3 μmol/L):
Corticosterone (10 mnol/L)+11β-HSD2 antisense or 11β-HSD2 nonsense oligomer
Corticosterone (10 nmol/L)+11β-HSD1 antisense or 11β-HSD1 nonsense oligomer
In 11-dehydrocorticosterone experiments with vehicle alone
11-dehydrocorticosterone (100 nmol/L)+11β-HSD1 Antisense or 11β-HSD1 nonsense oligomer

Antisense phosphorothioate oligonucleotides, targeted to block either 11β-HSD2 or 11β-HSD1 gene expression, were obtained from Research Genetics, Huntsville Ala. Antisense oligomers complementary to 20 bp sequences spanning the ribosome binding/translation start site were used. Oligomer sequences were: 5′-CAT AAC TGC CGT CCA ACA GC-3′ (SEQ ID NO. 2) for 11β-HSD1 Antisense and 5′-AGG CCA GCG CTC CAT GAC TT-3′ (SEQ ID NO 4) for 11β-HSD2 antisense. In control experiments the corresponding sense sequence was used as the nonsense oligomer. Antisense and nonsense oligomers were added directly to each well at 20 μg/10:1 sterile H₂O per well for a final concentration of 3 μmol/L.

For contraction measurements, aortic rings were suspended by tungsten wires with 1 g of tension and placed in a vessel bath containing serum free DMEM/F12 media at 37° C. aerated with 95% O₂-5% CO, at pH 7.4. Vessels were equilibrated for 20 minutes and then tested with phenylephrine (1 nmol/L-10 μmol/L). Although phenylephrine is structurally not a catecholamine, it is considered to be a functional catecholamine as it activates both α and β adrenoceptors. Due to its favorable stability characteristics, it is widely used as a catecholamine substitute in experiments of this nature. The intensity of contraction was assessed by use of a Narishige micromanipulator and model FT03 force transducer (Grass Instrument Co. West Warwick, R.I.). Measurements were recorded on computer using the Labview 4.1 Virtual Instrument System (National Instruments, Austin, Tex.). Adhering to this protocol, test vessel viability by demonstrating the ability of the vessel to vigorously contract when exposed to known vasoconstrictors and relax back to baseline after treatment with acetylcholine.

Results: Effect of 11β-HSD Antisense On Vascular Contractile Response

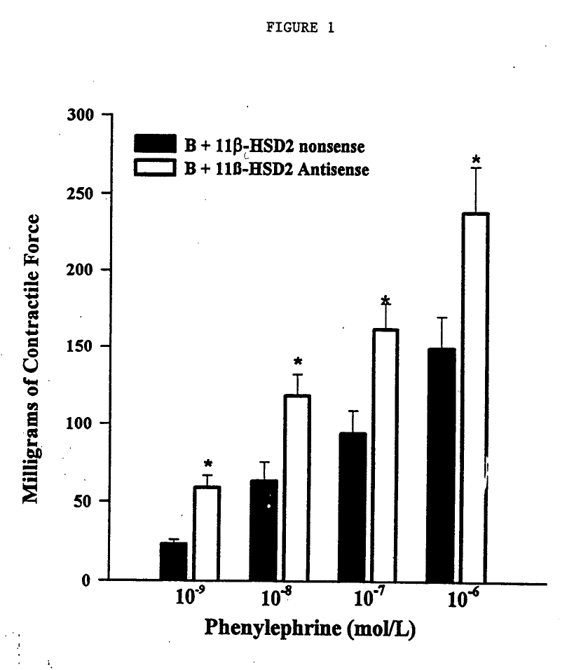

Experiments were carried out to determine whether specific 11β-HSD2 antisense oligomers affect the contractile response of vascular rings. Rat aortic rings, with endothelium intact, were incubated for 24 hours with corticosterone (10 nmol/L) and either specific 11β-HSD2 antisense oligomers (3 μmol/L) or nonsense oligomers (3 (μmol/L). Following incubation, the contractile responses to graded concentrations of phenylephrine were determined. Previously, it had been demonstrated that the incubation of aortic rings with corticosterone resulted in amplified contractile responses to graded concentrations of phenylephrine compared to controls. The exposure of rings to corticosterone together with 11β-HSD2 antisense demonstrated a statistically significant increase in the contractile response to all concentrations (1, 10, 100 nmol/L and 1 μmol/L) of phenylephrine (FIG. 1).

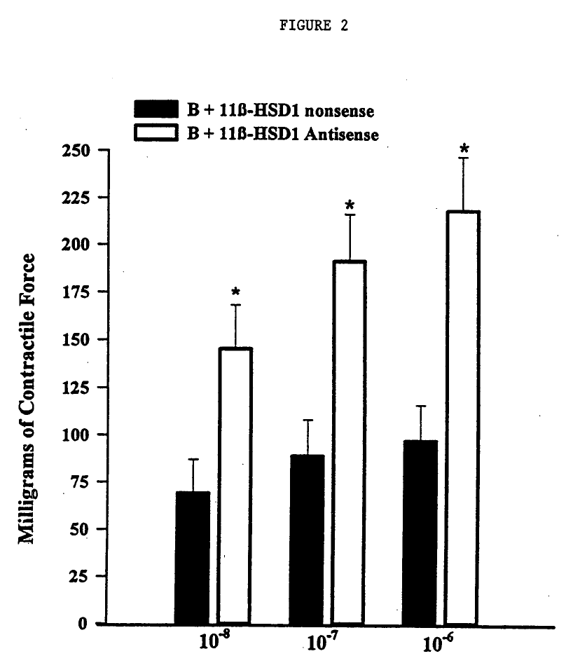

In the rat, both vascular endothelial and smooth muscle cells contain 11β-HSD1. Even though this isoform operates mainly as a reductase under physiologic conditions, it was examined if 11β-HSD1 antisense oligomers had an effect on the ability of corticosterone to amplify the contractile responses to phenylephrine in vascular tissue. Rings were incubated for 24-hours with corticosterone (10 nmol/L) and either 11β-HSD1 antisense oligomers (3 μmol/L) or nonsense oligomers (3 μmol/L). In rings treated with 11β-HSD1 antisense the contractile responses to all concentrations of phenylephrine (10 nmol/L, 100 nmol/L and 1 μmol/L) were significantly increased compared to rings treated with corticosterone and nonsense oligomers (FIG. 2).

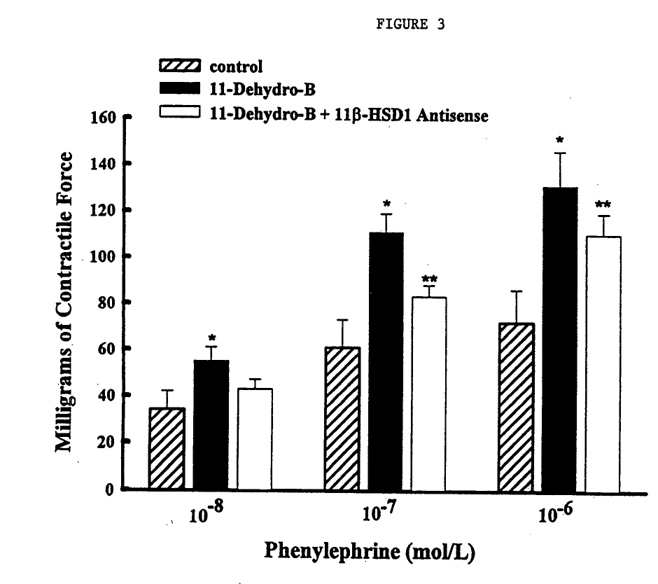

In rat vascular tissue, 11β-HSD1 acts predominantly as a reductase metabolizing inactive 11β-dehydro-glucocorticoid back to the active parent hormone. 11-dehydro-corticosterone (just like corticosterone) also amplifies the contractile responses to phenylephrine in rat aortic rings (FIG. 3). In the rat, 11β-HSD1 is present in both vascular endothelial and smooth muscle cells and under physiological conditions this enzyme functions predominantly as a reductase.

Furthermore, the effect of 11β-HSD1 antisense oligomers on the ability of 11-dehydro-corticosterone to amplify the contractile responses to phenylephrine was studied. Rings were incubated for 24 hours with 11-dehydro-corticosterone (100 nmol/L) and either 11β-HSD1 antisense (3 μmol/L) or nonsense (3 μmol/L) oligomers. 11β-HSD1 antisense oligomers attenuated the ability of 11β-dehydro-corticosterone to amplify the contractile response to all concentrations of phenylephrine compared to 11-dehydro-corticosterone plus 11β-HSD1 nonsense oligomers. Statistically significant decreases were observed at 100 nmol/L and 1 μmol/L phenylephrine (FIG. 3).

In aortic rings incubated (24-hours) with corticosterone (10 nmol/L) and 11β-HSD2 antisense (3 μmol/L), the contractile response to graded concentrations of phenylephrine (PE: 10 nmol/L-1 μmol/L) were significantly (P<0.05) increased compared to rings incubated with corticosterone and 11β-HSD2 nonsense. 11β-HSD1 antisense oligomers also enhanced the ability of corticosterone to amplify the contractile response to phenylephrine.

Discussion

Earlier experiments showed that inhibitors of 11β-HSD dehydrogenase activity enhance the ability of corticosterone to amplify the vasoconstrictive actions of phenylephrine and angiotensin II in rat aorta. The examples show that a specific 11β-HSD2 antisense oligomer also enhances the ability of corticosterone to amplify the contractile responses of catecholamines. Since 11β-HSD2 appears to exist only in endothelial cells, this observation supports a role for the action of glucocorticoids in affecting endothelial cell function. Although 11β-HSD1 acts predominantly as a reductase in vascular tissue, 11β-HSD1 antisense oligomers also enhanced the ability of corticosterone to amplify the contractile effects of phenylephrine in rat aortic rings. This observation suggests that 11β-HSD1-dehydrogenase, in addition to 11β-HSD2, also operates to protect GR and MR from over-activation by glucocorticoids in vascular tissue. Further experiments to determine whether antisense oligomers down-regulate mRNA and protein expression of their respective 11β-HSD isoform under conditions in which they enhance contractile responses in aortic rings will be done. Using a similar protocol to the one described here, it has been shown using RT-PCR analysis, that 11β-HSD2 antisense and 11β-HSD1 antisense down-regulate the expression of their respective enzyme isoforms in cultured rat vascular endothelial and smooth muscle cells.

The example confirms that 11-dehydro-corticosterone also amplifies the contractile actions of catecholamines in rat aortic rings. Since 11-dehydro-glucocorticoids do not bind to GR (or MR) to any major extent, it is proposed that 11-dehydro-corticosterone is metabolized back to corticosterone by 11β-HSD1-reductase in vascular smooth muscle and/or endothelial cells. This hypothesis is supported by the discovery that 11-keto-progesterone, a specific inhibitor of 11β-HSD1-reductase activity (backward reaction), diminished the ability of 11-dehydro-corticosterone to amplify the contractile effects of phenylephrine and decreased the metabolism of 11-dehydro-corticosterone back to corticosterone. The examples also demonstrate that 11β-HSD1 antisense oligomer also attenuates the ability of 11-dehydro-corticosterone to amplify the contractile responses of phenylephrine indicating that the down-regulation of 11β-HSD1 gene expression can affect the regeneration of active glucocorticoid (from 11-dehydro-glucocorticoid) in vascular tissue. Indeed, the examples show that 11β-HSD1 antisense can significantly reduce the metabolism of 11-dehydro-corticosterone back to corticosterone in aortic ring preparations.

### Example 2

### Metabolism of Corticosterone And 11-Dehydro-Corticosterone In Vascular Tissue

Experimental

The effects of 11β-HSD1 and 11β-HSD2 antisense on the inter-conversion of ³H-corticosterone and ³H-11-dehydro-corticosterone by aortic rings was also determined. Rings (2-3 mm) obtained in a similar manner as those in the contraction studies, were incubated in 1 ml DMEM/F12 media containing 1% FBS at 37° C. under 95% O₂-5% CO₂ in 24-well culture plates. Rings were incubated for 24 hours with:
(i) ³H-corticosterone (10 nmol/L)±11β-HSD2 or 11β-HSD1 antisense (3 μmol/L); control groups received nonsense oligomers. The amount of ³H-11-dehydro-corticqsterone in the incubation medium after 24 hrs was then measured. The effects of 11β-HSD1 antisense/nonsense were measured in quadruplicate (n=6 aortic rings per well) and the effects of 11β-HSD2 antisense/nonsense in duplicate (n=8 aortic rings per well),
(ii) ³H-11-dehydro-corticosterone (10 nmol/L)±11β-HSD1 antisense (3 μmol/L); this experiment was performed in duplicate (n=10 aortic rings per well). Control groups were incubated with the appropriate nonsense oligomer. ³H-corticosterone in the incubation medium after 24 hrs was then measured. In this experiment, aortic rings were also analyzed for ³H-corticosterone content. Rings from duplicate incubations (total n=20) were blotted dry, pooled and homogenized in 50 % methanol using a Polytron. The homogenates were then centrifuged, extracted as below using Sep-Paks and injected onto a HPLC system for analysis.

Incubation media was collected, ran through a Sep-Pak and eluted with 3 mls of methanol, the eluate was then dried under nitrogen and reconstituted in 500:1 methanol. The aortic rings were dried and weighed. The steroids present in the eluate were separated by high-pressure liquid chromatography with a Dupont Zorbax C8 column eluted at 44° C. at a flow rate of 1 mL/min using 55% methanol for 10 minutes. Steroids were observed by monitoring radioactivity on-line with a Packard Radiomatic Flo-One/Beta Series A-500 counter connected to a Dell Optiflex 425 S/L computer. Corticosterone and 11-dehydro-corticosterone were identified by comparing their retention times with that of known standards.

Corticosterone and phenylephrine were obtained from Sigma (St Louis, Mo.), 11-dehydrocorticosterone from Research Plus (Bayonne, N.J.) and ³H-steroids from New England Nuclear (Boston, Mass.). Where appropriate, data were expressed as mean ±SE and analyzed using ANOVA and the Student's t test with Bonferroni modification. P values of less than 0.05 are considered significant.

Results: Effects of 11β-HSD Antisense On Steroid Metabolism

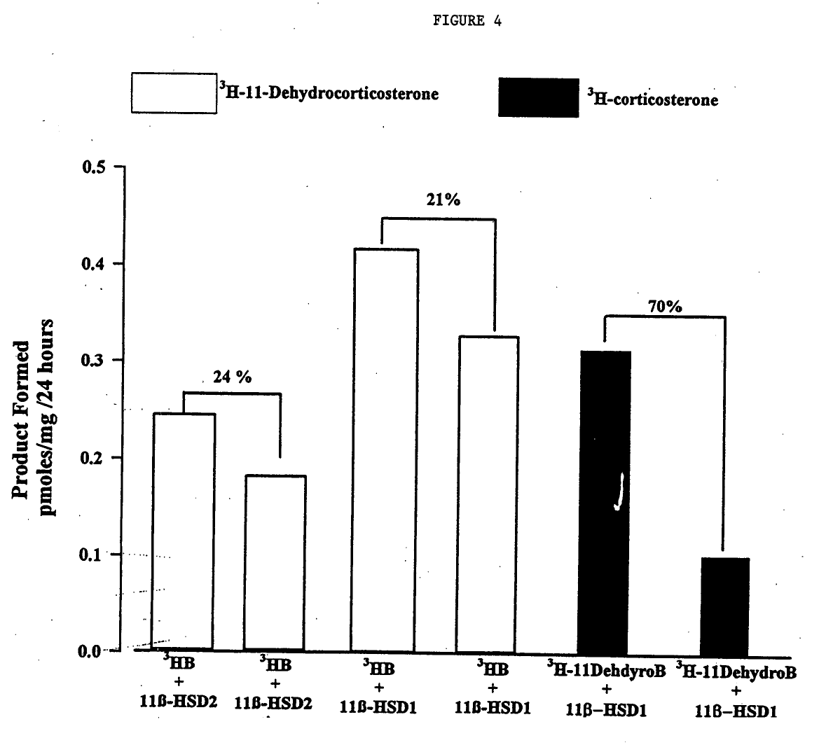

A series of experiments were then conducted to test whether 11β-HSD2 and 11β-HSD1 antisense oligomers did affect the enzymatic conversion of corticosterone and 11-dehydrocorticosterone. In experiments in which aortae were taken from rats (n=4) and 6 rings cut from each aorta were incubated for 24 hrs with ³H-corticosterone (10 nM) plus 11β-HSD1 antisense (3 μM), the conversion of corticosterone to 11-dehydrocorticosterone was 21% lower than in aortic rings incubated with corticosterone and 11β-HSD1 nonsense oligomers (FIG. 4). In a further two experiments, aortae were taken from rats (n=2) and 8 aortic rings cut from each. Aortic ring preparations incubated for 24 hrs with corticosterone and 11β-HSD2 antisense (3 μM), demonstrated a 24% reduction in the conversion of corticosterone to 11-dehydrocorticosterone compared to aortic rings incubated with corticosterone and 11β-HSD2 nonsense (FIG. 4).

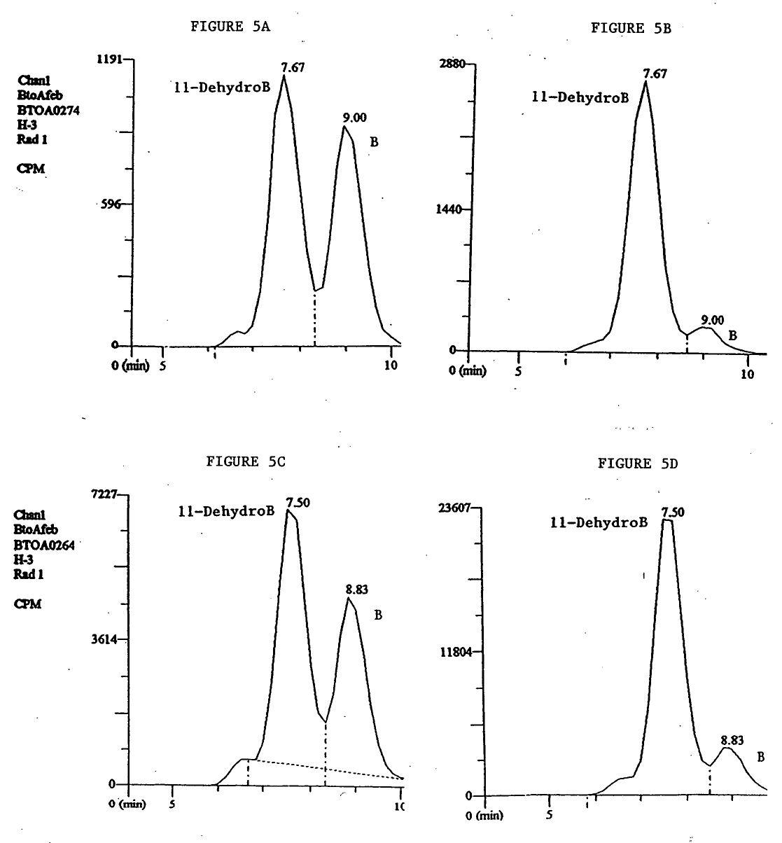

To determine the effects of 11β-HSD1 antisense on 11β-HSD1-reductase activity rat aortae were taken from rats (n=2) and 10 aortic rings cut from each. These aortic rings were then incubated for 24 hours with ³H-11-dehydrocorticosterone and either 11-HSD1 antisense or nonsense and the production of corticosterone was measured. The production of ³H-corticosterone was markedly reduced in rings incubated with 11β-HSD1 antisense compared to rings incubated with 11β-HSD1 nonsense oligomers (FIG. 4, representative HPLC chromatograms from these experiments are also shown in FIG. 5). Thus, 11β-HSD1 antisense profoundly diminished the ability of the rat aortic rings to-metabolize 11-dehydro-corticosterone back to corticosterone. The aortic ring tissue in these experiments was also pooled (n=20) and analyzed for steroid content. The amount of radioactivity in the tissue was approximately 2-3% of the total radioactivity in the incubation media. The production of ³H-corticosterone in aortic rings incubated with 11β-HSD1 antisense was again markedly lower that that in rings incubated with 11β-HSD1 nonsense oligomers (see HPLC chromatograms, FIGS. 5A-5D). The levels of ³H-11-dehydrocorticosterone metabolism measured in the incubate and in the aortic tissue were very similar (FIGS. 5A-5D). This indicates that measuring steroid content in the media does not under-represent the level of steroid metabolism in the tissue compartment.

Discussion

In this example, experiments were undertaken to determine whether antisense oligomers could affect 11β-HSD enzyme activity and, indeed, it has been demonstrated that 11β-HSD2 and 11β-HSD1 antisense caused moderate reductions (24 and 21% respectively) in the metabolism of corticosterone. These reductions in metabolism translate to relatively small increases in residual corticosterone levels in the aortic ring tissue that would not appear to account for the relatively large increases in phenylephrine-induced vasoconstriction observed in the contractile studies. However, glucocorticoids have been reported to not only amplify the contractile effects of catecholamines in vascular tissue but to also diminish the effects of certain vasorelaxation pathways (glucocorticoids decrease nitric oxide and prostaglandin I₂ synthesis); such actions would serve to further enhance the effects of glucocorticoids on increasing catecholamine-induced vasoconstriction and may explain how small changes in glucocorticoid levels can have profound effects on vascular tone.

In addition, 11β-HSD2 and 11β-HSD1 antisense also decreased the metabolism of corticosterone to 11-dehydro-corticosterone. 11-dehydro-corticosterone (100 nmol/L) also amplified the contractile response to phenylephrine in aortic rings (P<0.01), most likely due to the generation of active corticosterone by 11β-HSD1-reductase; this effect was significantly attenuated by 11β-HSD1 antisense. 11β-HSD1 antisense also caused a marked decrease in the metabolism of 11-dehydro-corticosterone back to corticosterone by 11β-HSD1- reductase. These findings underscore the importance of 11β-HSD2 and 11β-HSD1 in regulating local concentrations of glucocorticoids in vascular tissue. They also indicate that decreased 11β-HSD2 activity may be a possible mechanism in hypertension and other blood pressure associated disorders and that 11β-HSD1-reductase may be a possible target for anti-hypertensive therapy.

The results of these examples underscore the importance of 11β-HSD2 in regulating the access of glucocorticoids to GR and/or MR in vascular tissue and suggest that 11β-HSD1-dehydrogenase may also play a role in protecting GR and MR in this tissue. In addition, they suggest that the antisense oligomers used in these experiments down-regulate 11β-HSD gene expression and decrease glucocorticoid metabolism in vascular tissue, an effect leading to increased vascular responsiveness to catecholamines.

The examples also demonstrate that both 11β-HSD2 and 11β-HSD1 regulate local glucocorticoid concentrations in vascular tissue with 11β-HSD2 and 11β-HSD1-dehydrogenase working to decrease- and 11β-HSD1-reductase increase the amount of glucocorticoid that can access GR and MR in vascular smooth muscle. Physiological concentrations of both free corticosterone and 11-dehydrocorticosterone are similar over the course of the day in rodents. Therefore, significant quantities of not only glucocorticoid, but also of 11-dehydro-glucocorticoid are available for conversion back to the glucocorticoid. Since glucocorticoids amplify catecholamine and angiotensin II pressor responses and may inhibit the effects of some vasorelaxant pathways, a possible mechanism that may increase vascular tone and induce hypertension includes a decrease in 11β-HSD2 activity. Interestingly, many patients with essential hypertension also demonstrate decreased 11β-HSD2 activity as assessed by altered plasma and urinary cortisol/cortisone ratios. Moreover, the plasma half-life of 11α-³H-cortisol is prolonged in patients with essential hypertension consistent with the idea that 11β-HSD2 activity is diminished in this condition. The present work also suggests that since 11β-HSD1 reductase generates active glucocorticoid in vascular tissue, a possible therapeutic target in the treatment of hypertension could be the specific inhibition of 11β-HSD1 reductase activity.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

All patents, patent applications, and literature references cited herein or in Appendix A are hereby expressly incorporated by reference.

1. A method for treating apparent adrenal insufficiency in a subject, comprising administering to said subject an effective amount of an 11β-HDSD1 dehydrogenase inhibitor or a 11β-HSD2 dehydrogenase inhibitor, such that said subject is treated for said apparent adrenal insufficiency.

2. The method of claim 1, wherein said subject is undergoing surgery.

3. The method of claim 1, wherein said subject is being treated for sepsis or hyponatremia.

4. The method of claim 1, wherein an 11β-HSD1 dehydrogenase inhibitor is administered in combination with an 11β-HSD2 dehydrogenase inhibitor to said subject.

5. The method of claim 1, wherein said 11β-HSD1 dehydrogenase inhibitor is 3α, 5α-reduced-11μ-OH-progesterone, 3α, 5α-reduced-11β-OH-testosterone, 3α, 5α-reduced-11β-OH-androstendione, 3α, 5α-reduced-11β-OH-pregnenolone, 3α, 5α-reduced-11β-OH-dehydro-epiandrostenedione, 3α, 5α-reduced-corticosterone, 3α, 5α-reduced-aldosterone, 3α, 5α-reduced-pregnenolone, 3α, 5α-reduced-dehydro-epiandrostenedione, 3α, 5β-reduced-progesterone, 3α, 5β-reduced testosterone, deoxy-corticosterone, 11β-OH progesterone, 11β-OH testosterone, 11β-OH-pregnenolone, 11β-OH-dehydro-epiandrostenedione, 3α, 5α-reduced-progesterone, 3α, 5α-reduced testosterone, 3α, 5α-reduced-chenodeoxycholic acid, glycyrrhetinic acid, carbenoxolone, 3α,5β-reduced deoxycorticosterone, 3α, 5β-reduced-chenodeoxycholic acid, 3α, 5β-reduced progesterone, 3α, 5α-reduced deoxycorticosterone, or a pharmaceutically acceptable prodrug or salt thereof.

6. The method of claim 1, wherein said 11β-HSD2 dehydrogenase inhibitor is a nucleic acid, 3α, 5α-reduced-11β-OH-progesterone, 3α, 5α-reduced-11β-OH-testosterone, 3α, 5α-reduced-11β-OH-androstenedione, 3α, 5α-reduced-11-keto-progesterone, 3α, 5α-reduced-11-dehydro-corticosterone, 3α, 5α-reduced-corticosterone, 3α, 5α-reduced aldosterone, 3α, 5α-reduced deoxycorticosterone, 11β-OH-progesterone, 11β-OH-testosterone, 11-keto-progesterone, 5α-dihydro-corticosterone, 5α-dihydro-corticosterone, glycyrrhetinic acid, carbenoxolone or a pharmaceutically acceptable prodrug or salt thereof.

7. The method of claim 1, wherein said 11β-HSD1 dehydrogenase inhibitor or said 11β-HSD2 dehydrogenase inhibitor is administered to said subject's kidney.

8. A method for increasing the half-life of glucocorticoid drugs in a subject, comprising administering to said subject an effective amount of a 11β-HSD2 dehydrogenase inhibitor in combination with said glucocorticoid drug, such that the half life of said glucocorticoid drug in said subject is increased.

9. The method of claim 8, wherein said glucocorticoid drug is an 11-keto glucocorticoid drug.

10. The method of claim 9, wherein said drug is selected from the group consisting of prednisone, 9α-fluorocortisone, 9α-fluoro-16α-hydroxyprednisone, and dexamethasone.

11. The method of claim 8, wherein said 11β-HSD2 dehydrogenase inhibitor is a nucleic acid, 3α, 5α-reduced-11β-OH-progesterone, 3α, 5α-reduced-11β-OH-testosterone, 3 α, 5α-reduced-11β-OH-androstenedione, 3α, 5α-reduced-11-keto-progesterone, 3α, 5α-reduced-11-dehydro-corticosterone, 3α, 5α-reduced-corticosterone, 3α, 5α-aldosterone, 11β-OH-progesterone, 11β-OH-testosterone, 11-keto-progesterone, 5α-dihydro-corticosterone, 5α-dihydro-corticosterone, 3α, 5α-reduced deoxycorticosterone, glycyrrhetinic acid, carbenoxolone or a pharmaceutically acceptable prodrug or salt thereof.

12. The method of claim 8, wherein said 11β-HSD2 inhibitor is administered to said subject's kidney.


SEQUENCE LISTING (7 sequences)

SEQ ID NO: 1 - DNA, 1375 bp, Homo sapiens
attcagaggctgctgcctgcttaggaggttgtagaaagctctgtaggttctctctgtgtgtcctacaggagtcttcaggccagctccctgtcggatggcttttatgaaaaaatatctcctccccattctggggctcttcatggcctactactactattctgcaaacgaggaattcagaccagagatgctccaaggaaagaaagtgattgtcacaggggccagcaaagggatcggaagagagatggcttatcatctggcgaagatgggagcccatgtggtggtgacagcgaggtcaaaagaaactctacagaaggtggtatcccactgcctggagcttggagcagcctcagcacactacattgctggcaccatggaagacatgaccttcgcagagcaatttgttgcccaagcaggaaagctcatgggaggactagacatgctcattctcaaccacatcaccaacacttctttgaatctttttcatgatgatattcaccatgtgcgcaaaagcatggaagtcaacttcctcagttacgtggtcctgactgtagctgccttgcccatgctgaagcagagcaatggaagcattgttgtcgtctcctctctggctgggaaagtggcttatccaatggttgctgcctattctgcaagcaagtttgctttggatgggttcttctcctccatcagaaaggaatattcagtgtccagggtcaatgtatcaatcactctctgtgttcttggcctcatagacacagaaacagccatgaaggcagtttctgggatagtccatatgcaagcagctccaaaggaggaatgtgccctggagatcatcaaagggggagctctgcgccaagaagaagtgtattatgacagctcactctggaccactcttctgatcagaaatccatgcaggaagatcctggaatttctctactcaacgagctataatatggacagattcataaacaagtaggaactccctgagggctgggcatgctgagggattttgggactgttctgtctcatgtttatctgagctcttatctatgaagacatcttcccagagtgtccccagagacatgcaagtcatgggtcacacctgacaaatggaaggagttcctctaacatttgcaaaatggaaatgtaataataatgaatgtcatgcaccgctgcagccagcagttgtaaaattgttagtaaacataggtataattaccagatagttatattaaatttatatcttatatataataatatgtgatgattaatacaatattaattataataaaggtcacataaactttataaattcataactggtagctataacttgagcttattcaggatggttctttaaaccataaactgtacaatg

SEQ ID NO: 2 - DNA, 20 bp, Artificial Sequence
  oligomer sequence
cataactgccgtccaacagc

SEQ ID NO: 3 - DNA, 1873 bp, Homo sapiens
cgcgccccaggccggtgtacccccgcactccgcgccccggcctagaagctctctctccccgctccccggcccggcccccgccccgccccgccccagcccgctgggccgccatggagcgctggccttggccgtcgggcggcgcctggctgctcgtggctgcccgcgcgctgctgcagctgctgcgctcagacctgcgtctgggccgcccgctgctggcggcgctggcgctgctggccgcgctcgactggctgtgccagcgcctgctgcccccgccggccgcactcgccgtgctggccgccgccggctggatcgcgttgtcccgcctggcgcgcccgcagcgcctgccggtggccactcgcgcggtgctcatcaccggctgtgactctggttttggcaaggagacggccaagaaactggactccatgggcttcacggtgctggccaccgtattggagttgaacagccccggtgccatcgagctgcgtacctgctgctcccctcgcctaaggctgctgcagatggacctgaccaaaccaggagacattagccgcttgctagagttcaccaaggcccacaccaccagcaccggcctgtggggcctcgtcaacaacgcaggccacaatgaagtagttgctgatgcggagctgtctccagtggccactttccgtagctgcatggaggtgaatttctttggcgcgctcgagctgaccaagggcctcctgcccctgctgcgcagctcaaggggccgcatcgtgactgtggggagcccagcgggggacatgccatatccgtgcttgggggcctatggaacctccaaagcggccgtggcgctactcatggacacattcagctgtgaactccttccctggggggtcaaggtcagcatcatccagcctggctgcttcaagacagagtcagtgagaaacgtgggtcagtgggaaaagcgcaagcaattgctgctggccaacctgcctcaagagctgctgcaggcctacggcaaggactacatcgagcacttgcatgggcagttcctgcactcgctacgcctggccatgtccgacctcaccccagttgtagatgccatcacagatgcgctgctggcagctcggccccgccgccgctattaccccggccagggcctggggctcatgtacttcatccactactacctgcctgaaggcctgcggcgccgcttcctgcaggccttcttcatcagtcactgtctgcctcgagcactgcagcctggccagcctggcactaccccaccacaggacgcagcccaggacccaaacctgagccccggcccttccccagcagtggctcggtgagccatgtgcacctatggcccagccactgcagcacaggaggctccgtgagccttggttcctccccgaaaacccccagcattacgatcccccaagtgtcctggaccctggcctaaagaatcccacccccacttcatgcccactgccgatgcccaatccaggcccggtgaggccaaggtttcccagtgagcctctgcgcctctccactgtttcatgagcccaaacaccctcctggcacaacgctctaccctgcagcttggagaactccgctggatgggagtctcatgcaagacttcactgcagcctttcacaggactctgcagatagtgcctctgcaaactaaggagtgactaggtgggttggggaccccctcaggattgtttctcggcaccagtgcctcagtgctgcaattgagggctaaatcccaagtgtctcttgactggctcaagaattagggccccaactacacacccccaagccacagggaagcatgtactgtacttcccaattgccacattttaaataaagacaaatttttatttcttcta

SEQ ID NO: 4 - DNA, 20 bp, Artificial Sequence
  oligomer sequence
aggccagcgctccatgactt

SEQ ID NO: 5 - DNA, 1755 bp, Homo sapiens
  CDS (61)...(1587)
gagttgccacagctcttctactccactgctgtctatcttgcctgccggcacccagccaccatgtgggagctcgtggctctcttgctgcttaccctagcttatttgttttggcccaagagaaggtgccctggtgccaagtaccccaagagcctcctgtccctgcccctggtgggcagcctgccattcctccccagacatggccatatgcataacaacttcttcaagctgcagaaaaaatatggccccatctattctgttcgtatgggcaccaagactacagtgattgtcggccaccaccagctggccaaggaggtgcttattaagaagggcaaggacttctctgggcggcctcaaatggcaactctagacatcgcgtccaacaaccgtaagggtatcgccttcgctgactctggcgcacactggcagctgcatcgaaggctggcgatggccacctttgccctgttcaaggatggcgatcagaagctggagaagatcatttgtcaggaaatcagtacattgtgtgatatgctggccacccacaacggacagtccatagacatctcctttcctgtcttcgtggcggtaaccaatgtcatctccttgatctgcttcaatacctcctacaagaatggggaccctgagttgaatgtcatacagaattacaatgaaggcatcatagacaacctgagcaaagacagcctggtggacctagtcccctggttgaagattttccccaacaaaaccctggaaaaattaaagagccatgttaaaatacgaaatgatctgctgaataaaatacttgaaaattacaaggagaaattccggagtgactctatcaccaacatgctggacacactgatgcaagccaagatgaactcagataatggcaatgctggcccagatcaagattcagagctgctttcagataaccacattctcaccaccataggggacatctttggggctggcgtggagaccaccacctctgtggttaaatggaccctggccttcctgctgcacaatcctcaggtgaagaagaagctctacgaggagattgaccagaatgtgggtttcagccgcacaccaactatcagtgaccgtaaccgtctcctcctgctggaggccaccatccgagaggtgcttcgcctcaggcccgtggcccctatgctcatcccccacaaggccaacgttgactccagcatcggtgagtttgctgtggacaagggcacagaagttatcatcaatctgtgggcgctgcatcacaatgagaaggagtggcaccagccggatcagttcatgcctgagcgtttcttgaatccagcggggacccagctcatctcaccgtcagtaagctatttgcccttcggagcaggacctcgctcctgtataggtgagatcctggcccgccaggagctcttcctcatcatggcctggctgctgcagaggttcgacctggaggtgccagatgatgggcagctgccctccctggaaggcatccccaaggtggtctttctgatcgactctttcaaagtgaagatcaaggtgcgccaggcctggagggaagcccaggctgagggtagcacctaaaggctgtaactcacagcccctgtccaccctatgtggccccacaacacagatttagagatacaaccccccacccttctccgccattcttccctactcccaacccactctgccttctttttcagcttgtggcaatgccagtgatgtgcataaacagttttttttttttcc

SEQ ID NO: 6 - DNA, 1006 bp, Homo sapiens
gtcatttgcaaagggccatccttcccaacagagacaagcgcctgctccctgacttccttggccttcccaggatctgactcttccctttcctttcaccccagatatcgcaggcaccagtgacaagtgggaaaagctggagaaggacattctggaccacctccccgctgaggtacaggaagactacggccaggactacatcttagcacagcggaatttcctcctattgatcaactcgttagccagcaaggacttctctccggtgctgcgggacatccagcatgctatcttggcgaagagcccttttgcctattacacgccagggaaaggcgcttacttgtggatctgccttgctcactatttgcctattggcatatatgattactttgctaaaagacattttggccaagacaagcccatgcccagagctctaagaatgcctaactacaagaaaaaggccacctaggcaatggaagccctcaaagaagtcggaatgtcatagtcttgaaatgaaagggaaactgggaaactgggtttctcattaaagttgtttcccactctgtattctctgtgaaattcattgattgaatagtactttgtagtgagaaggctggagaatagcaacagcctcatggagaatgttaattgagttcctactatgcaccagatacattacaggtattctttcatctagttctcatcagagaattgaggtgggtatctttgcccctacttaatataatcagagagatttcttgtcacactctagcaaaaggcagggccagaattcaaatcctgagttggactccactgcctgccaagagaagaggaagagaaagagagagacaaggagaaagagagagggaaggaaaggaggaggcagaaatggcaagggcaacagtccaatggatcttgcaagtcttcctcactgtgcattgatattccgtgactactgcacccaaggtattggattcatggtaaatcatcagggcttcgttctcttccttcc

SEQ ID NO: 7 - protein, 387 aa, Homo sapiens
MSTFFSDTAWICLAVPTVLCGTVFCKYKKSSGQLWSWMVCLAGLCAVCLLILSPFWGLILFSVSCFLMYTYLSGQELLPVDQKAVLVTGGDCGLGHALCKYLDELGFTVFAGVLNENGPGAEELRRTCSPRLSVLQMDITKPVQIKDAYSKVAAMLQDRGLWAVINNAGVLGFPTDGELLLMTDYKQCMAVNFFGTVEVTKTFLPLLRKSKGRLVNVSSMGGGAPMERLASYGSSKAAVTMFSSVMRLELSKWGIKVASIQPGGFLTNIAGTSDKWEKLEKDILDHLPAEVQEDYGQDYILAQRNFLLLINSLASKDFSPVLRDIQHAILAKSPFAYYTPGKGAYLWICLAHYLPIGIYDYFAKRHFGQDKPMPRALRMPNYKKKAT